(19) United States
(12) Patent Application Publication
(10) Pub. No.: US 2012/0225111 A1
(43) Pub. Date: Sep. 6, 2012

(54) PRODUCT FOR THE MANAGEMENT OF INFLAMMATIONS, PRESSURE SORES AND/OR ORAL SORES (APHTHAS) AS WELL AS THE USE OF SUCH PRODUCT

(75) Inventor: Gert Hartmut Scholz, Neuwied (DE)

(73) Assignee: Lohmann & Rauscher GmbH & Co. KG, Neuwied (DE)

(21) Appl. No.: 11/936,014

(22) Filed: Nov. 6, 2007

(30) Foreign Application Priority Data
Nov. 6, 2006 (EP) 06023033.1

(51) Int. Cl. A61L 15/16

(52) U.S. Cl. 424/444

(57) ABSTRACT

The invention relates to a product for the management of inflammations, pressure sores and/or oral sores (aphthas) which is made predominantly, and preferably completely, from a bioresorbable material, with the bioresorbable material exhibiting a flexible and/or compressible pad element which adheres to the mucosa, and in particular being designed completely as a pad element.

### CROSS REFERENCE TO RELATED APPLICATIONS

The present application claims international priority under 35 U.S.C. §119 to co-pending European Patent Application No. 06023033.1 filed Nov. 6, 2006, entitled “Produkt zur Versorgung von Entzuendungen, Druckstellen und/oder Aphthen im Oralbereich Sowie Verwendung eines Solchen Produktes” the entire content and disclosure of which is hereby incorporated by reference in its entirety.

### TECHNICAL FIELD

The invention relates to a product for the management of inflammations, pressure sores and/or oral sores (aphthas) which is made predominantly, and preferably completely, from a bioresorbable material as well as to the use of such a product.

### BACKGROUND

Symptoms in the oral region can have various causes. For example, the oral mucosa is more prone to problems if it is overly dry. Sometimes this condition occurs with increasing age, but it can also occur as a side effect of certain medications. Inflammations of the oral mucosa can also be due to a weakened immune system. Another possible cause of problems in the oral region is vitamin deficiency or allergic reactions. In this respect, frequent inflammations of the oral mucosa may be an indication of a deficiency of certain vitamins or iron. Allergic reactions to foods or drugs are also possible causes.

Especially frequent causes of problems in the oral region are dental prostheses. Especially in cases where new partial or complete dentures are fitted to the upper and lower jaw, there are often long periods of adjustment lasting several weeks, in the course of which pain and pressure sores can occur. Similar problems occur in the case of braces, which can rub against the oral mucosa and cause pressure sores and inflammations. During dental procedures as well, it is sometimes impossible to avoid pressure sores caused by the use of instruments. It often takes a few hours or several days for small wounds to develop in these places after the dental procedure. Injuries in the oral cavity can also be caused by the use of toothpicks, forks, bones, utensils or a broken tooth with sharp edges. It also happens, not infrequently, that people accidentally bite their cheeks, lips or tongue. While brushing one's teeth, even the use of a hard toothbrush can often lead to gum injuries, which initially go unnoticed. Furthermore, some inflammations of the oral mucosa have causes that are not clearly identifiable. Some possible causes are, for example, mycosis (oral candidiasis), virus infections (e. g. herpes virus infections) or infections caused by bacteria.

One of the most frequent conditions of the oral mucosa is the so-called aphthas (oral sores). Most of the time, oral sores are lentil-sized and painful mucosal changes surrounded by a red inflamed border. The places where they most frequently develop are the bottom of the mouth, the mucosa of the cheeks and tongue and the inside of the lips. Sometimes they are triggered by injuries, as described above, or they simply occur as an unpleasant concomitant phenomenon of infectious diseases. The real cause, however, has not been scientifically determined up to now.

A number of products for the treatment of the above-described inflammations, pressure sores and/or oral sores are known. These products are available in the form of solutions for rinsing, gels, sprays, troches or lozenges. They contain active agents such as benzalkonium or cetrimonium, chlorhexidine, cetyl pyridinium chloride, cresol, dichlorobenzyl alcohol or hexetidine. The active agent tyrothricin is an antibiotic which renders bacteria which cause inflammations innocuous. Furthermore, some herbal preparations which have a disinfecting effect and inhibit inflammations are known. These products also come in a variety of administration forms, such as, e. g., ointments, gels, sprays, solutions and tinctures. They contain, for example, extracts of medicinal plants, such as sage, chamomile, peppermint, Echinacea (coneflower), Sycygium aromaticum, myrrh bush and rhubarb. Peppermint and clove oil not only have antiseptic effects but also local anesthetic effects. Echinacea extracts activate the body's own immune system. Myrrh tinctures and rhubarb root extracts have an astringent effect and protect the mucosa.

Remarkable successes have been achieved with the known products for the management of inflammations, pressure sores and/or oral sores. But these products have to be used in high doses. Furthermore, in many cases, especially with problems in the area of prostheses or braces, reinflammations and/or recurrence of pressure sores are observed shortly after discontinuation of these products.

### SUMMARY

In view of the above-named problems in the state of the art, the invention is based on the technical problem of providing products for the management of inflammations, pressure sores and/or oral sores making it possible to achieve sustained therapeutic success using low doses.

In accordance with the invention, this problem is solved using a further development of the known products, characterized essentially in that the bioresorbable material includes a flexible and/or compressible pad element which adheres to the mucosa, and in particular, that it is designed completely as a pad element.

This invention is based on the insight that the problems in the state of the art with respect to the high dose required to achieve the desired therapeutic success are essentially due to the fact that the typically used solutions, gels or sprays spread in the entire oral cavity, making it necessary to use large quantities of the respective products to treat local inflammations or pressure sores. This problem can be eliminated if the bioresorbable material used to treat the inflammations, pressure sores and/or oral sores includes a pad element which adheres to the mucosa. Then it becomes possible to place the bioresorbable material selectively in the problematic places in the oral cavity. Using the products according to the invention, it is no longer necessary to use an excessively high quantity of the active substance.

In accordance with a further aspect of the invention, the bioresorbable material is implemented as a flexible and/or compressible pad element. This aspect of the invention is based on the insight that the rapid recurrence of problems in the area of complete prostheses and/or braces is caused by the local pressure exerted by these elements.

Using the flexible and/or compressible pad elements designed in accordance with the invention, this local pressure can be distributed over larger areas or it can be cushioned. In this way, the cause of the pressure sores and inflammations can be eliminated using the products in accordance with the invention.

With respect to the desired bioresorbability while simultaneously ensuring flexible and/or compressible properties, it has been shown to be expedient for the bioresorbable material to include a collagen, preferably mixed with at least one concomitant substance. Collagen can be provided in the form of compressible sponges which, when moistened, adhere to the oral mucosa, but do not include adhesive properties in the dry state. This facilitates the storage of the respective products and, at the same time, ensures the desired effects according to the invention with respect to the properties of adhering to the mucosa, flexibility and/or compressibility. Processes for the manufacture of the collagen sponges which are suitable for the manufacture of the products in accordance with the invention are described, for example, in DE 38 32 162 C2. The content disclosed in this patent specification with respect to the manufacture of collagen sponges is herewith incorporated in the present specification by explicit reference. The collagen in accordance with the invention may be a commercially available bovine, equine, porcine and/or marine collagen. In order to achieve the desired properties, for example to obtain the desired flexibility and compressibility, the bioresorbable material may comprise collagen and at least one other bioresorbable polymer. The other bioresorbable polymer may include an alginate, gellan, chitosan, chitin, k-carrageenan, φ-carrageenan, xanthan, carboxymethyl cellulose, hydroxyethyl cellulose and/or hydroxy ethyl propyl cellulose.

Among these substances, an alginate additive affects the release of polycation agents (e. g. biguanides such as polyhexanide). In this context, a continuous release of agents can be assumed. The CA alginate affects the mechanical load-bearing capacity and biodegradability. Furthermore, when Ca alginate is used, a positive benefit in the wound healing process has been reported. It should be emphasized that the addition of Ca alginate to collagen makes it possible to obtain an especially high reversible compressibility of the bioresorbable material.

Gellan can also have an effect on the release of biguanides. With respect to gellan, the literature also reveals descriptions of a wound-healing effect.

Chitosan has a bacteriostatic effect. Chitosan also has a hemostatic effect, which also has an effect on the combination with the bioresorbable matrix. An increase in the hemostatic and bacteriostatic effect is produced. In addition, chitosan constitutes a poor culture medium for microorganisms. For this reason, the use of chitosan prevents the rapid contamination of the collagen cushion, which by nature is a good culture medium for microorganisms. In a specific embodiment, chitosan is added in soluble form so that it exits from the collagen matrix and has a positive effect on the micro-climate in the oral cavity due to its film-forming properties. Chitin is insoluble in water. For this reason, chitin is advantageously used in the form of a copolymer of chitosan and chitin. This copolymer has approximately the same properties as chitosan.

In the combination with biguanides, such as e. g. polyhexanide, a change in the ionic charge of the collagen matrix produces an increased release of the active agent.

K-carrageenan is biodegradable and has a low solubility. For this reason, the collagen matrix can be adjusted to be more hydrophobic by adding k-carrageenan.

φ-carrageenan has a better solubility. Its effect is to improve the mouth feeling, in particular in the saliva-infused sample.

Xanthan is a highly effective thickening agent, which also produces an improved mouth feeling, especially in the saliva-infused collagen matrix.

The addition of carboxymethyl cellulose also produces an improved mouth feeling. Based on the polyanionic nature of the component, the release of biguanides is reduced. Contamination of the cushion is prevented. The release of the active agent (e. g. polyhexanide) is extremely low.

Hydroxyethyl cellulose and hydroxyethyl propyl cellulose in combination with collagen lead to an improved mouth feeling.

The concentration of the additional bioresorbable material is preferably 0.5 to 50% in relation to the dry weight of the bioresorbable carrier material (collagen). With a concentration of less than 0.5%, the desired effects are not obtained. With a concentration of 50% or more, the properties of the carrier material will be disadvantageously influenced. There will be a risk, for example, that the desired padding effect will no longer be achieved. This, however, does not apply to honey. Honey may also be added in higher concentrations without negatively influencing the properties of the carrier material.

In accordance with another embodiment of the invention, the bioresorbable material includes at least one concomitant substance which produces an increased adherence to the mucosa, an improved flexibility or clingability and/or an improved taste. This concomitant substance may include honey, propolis, aloe vera, panthenol, polyethylene glycol 200-2000, polyvinyl pyrrolidone, glycerin, chamomile extract, sage oil, peppermint oil, vitamins and/or provitamins. Honey may be added at a concentration of 60% or more, in relation to the dry content of the collagen matrix. Glycerin may be added in the amount of 0.1 to 50% in relation to the dry content of the collagen matrix. The chamomile extract is preferably added in the amount of 0.5 to 1.5%, specifically approximately 1%, relative to the dry content of the collagen matrix. Likewise, the sage oil is added preferably in the amount of 0.5 to 1.5%, preferably approx. 1%, relative to the dry content of the collagen matrix. Vitamin C can also be added in the amount of 0.5 to 1.5%, relative to the dry content of the collagen matrix, specifically in the amount of approx. 1%. Lemon extract is also added in the amount of 0.5 to 1.5% relative to the dry content of the collagen matrix, specifically in the amount of approx. 1%.

The concomitant substances mentioned so far—honey, glycerin, chamomile extract, sage oil, peppermint oil and vitamin C as well as lemon extract, cyclamate and other synthetic sweeteners—also serve to improve the taste, just like the orange extract which can also be used as a concomitant substance. In this context, cyclamate and other synthetic sweeteners are preferably used in the amount of 0.2 to 0.8%, specifically approx. 0.5%, relative to the dry content of the collagen matrix, whereas orange extract is added in the amount of 0.5 to 1.5%, specifically approx. 1%, relative to the dry content of the collagen matrix. Mint oils, specifically peppermint oil (Mentha×piperita) is expediently used in the amount of 0.5 to 1.5%.

An improved odor may be achieved by adding propolis in the amount of 0.5 to 1.5%, specifically approx. 1%, relative to the dry content of the collagen matrix; the same effect can also be achieved by adding extracts of citrus fruits in the amount of 0.5 to 1.5%, specifically approx. 1%, relative to the dry content of the collagen matrix.

An adhesion-changing effect is produced by means of glycerin, panthenol, polyethylene glycol 200-2000, polyvinyl pyrrolidone, aloe vera or other biodegradable polymers. In this context, the amount of glycerin is preferably 5 to 50%, relative to the dry content of the collagen matrix. Panthenol is advantageously added in the amount of 2 to 5%, relative to the dry content of the collagen matrix. Polyethylene glycol 200-2000 can be used in the amount of 1 to 5%, specifically approx. 3%, relative to the dry content of the collagen matrix. Polyvinyl pyrrolidone is advantageously used in the amount of 1.5 to 4.5%, specifically approx. 3%, relative to the dry content of the collagen matrix. The amount of aloe vera is preferably 1 to 3%, specifically approx. 2%, relative to the dry content of the collagen matrix.

Overall, it is expedient for the concentration of the concomitant substances to be in the range of 0.5 to 50%, relative to the dry weight of the carrier material (collagen). With a concentration of less than 0.5%, the desired effects are usually not obtained, while with a concentration of more than 50%, the properties of the collagen matrix may be disadvantageously influenced.

In another embodiment of the invention, the concomitant substance used to mix with the collagen may be contain an anesthetic and/or an antiseptic, with the antiseptic or the local anesthetic possibly being selected from the group of the esters or acid amides of paraamino benzoic acid. The antiseptic or the local anesthetic may be mixed with a small quantity of adrenalin to improve the active agent. Expediently, the anesthetic features ultracaine, lidocaine, mepivacaine, bupivacaine, prilocaine, articaine, procaine and/or tetracaine. The amount of ultracaine, lidocaine and/or mepivacaine is preferably approx. 0.1 to 0.3%, specifically approx. 0.2%, relative to the dry content of the collagen matrix. Bupivacaine is preferably added in the amount of 0.25 to 0.75%, specifically approx. 0.5%, relative to the dry content of the collagen matrix. The same applies to prilocaine. Articaine, procaine and tetracaine are now considered obsolete and are only used in the event of allergic reactions to the amine type.

The amount is expediently 0.5 to 1%, relative to the dry content of the collagen matrix.

Usually, local anesthetics are administered intramuscularly or applied superficially in the form of a gel to the gums or the oral mucosa. In this context, it is disadvantageous that the gel rapidly and extensively spreads in the oral cavity, especially when oral sores are treated, which most of the time develop on the lower lip. This leads to an unpleasant, extensive anesthetization in the oral cavity. This problem can be solved by using a biodegradable collagen matrix as a carrier material for the local anesthetic. In this way, the local anesthetic is continuously delivered to the oral mucosa. This results in the local anesthetization of only the spot that is intended to be anesthetized. The bioresorbable material used as a carrier, such as, for example, collagen, does not dissolve as quickly as an otherwise used gel, so that it becomes possible to obtain good anesthetization of the area to be treated over a longer period of time, using a comparatively smaller dose of the active agent.

In addition, another advantage of the use of a local anesthetic that should be emphasized is that not only children but also adults are uncomfortable with the use of an injection by the dentist. A local anesthetic used beforehand at the injection site leads to suppression of the pain on penetration of the needle, resulting in children not associating this moment with a bad experience any more. Traumatization is prevented. Although local anesthetics are available, they should not be used frequently with children under the age of 12. Furthermore, they do not have a very pleasant taste. A collagen pad releases only a fraction of the amount of the local anesthetic, especially when is it used under controlled conditions at the dentist's office. This makes it possible to use such a collagen matrix with children as well. Also, it can be assumed that a small piece of the collagen matrix with a local anesthetic, which, for example, has a sweet taste, will be accepted more readily by children than an ointment or a gel.

The concentration of the antiseptic or the local anesthetic is advantageously in the range of 0.2 to 2%, relative to the dry weight of the carrier material, for purposes of physiological effectiveness. In accordance with another aspect of the invention, the product in accordance with the invention may contain a concomitant substance including an antimicrobial compound. The antimicrobial compound may be taken from the spectrum of biguanides.

Specifically, it may be polyhexamethyl biguanide, hydrochloride, chlorohexidine, dihydrochloride, chlorohexidine diacetate, chlorohexidine D-gluconate, octenidine, taurolidine, cetylpyridinium hydrochloride and/or mixtures thereof.

Often wounds in the oral region are infected or the cause of the development of the wound is a microbiological colonization of the wound. One respective example is a traumatization or a pressure sore which is apt to transform into an infected wound at any time. But canker sores or the development of oral sores (aphthas) is by far more frequent in the dental area. These constitute wound infections.

As explained previously, one problem of the known products used for the treatment of these conditions is the local administration of antiseptic substances. A gel quickly spreads in the oral cavity and results in a negative effect on the micro-climate of the oral cavity. Due to this dispersion, the concentration of the active agent in the spot to be treated rapidly drops to an insufficiently low level. This results in an insufficient effectiveness of the antiseptic or no effectiveness at all. The same applies to gargle solutions or gels that are used to treat periodontosis. To treat periodontosis, a gel having an antimicrobial effect is placed into a fitting denture form, which, however, first has to be made. This creates especially high costs for the cost units. The effect obtained in this way is an antimicrobial action lasting over an extended period of time since the gel is prevented from spreading out through the denture form.

The biodegradable collagen matrix in accordance with the invention provides a solution to the above-mentioned issues caused by the spreading effect or the cost problems. By using such a matrix, the active agent is continuously released at the infected wound. Thanks to the firm structure of the carrier material or the collagen matrix, it is impossible for the cushion to spread out. This allows for the management of a wound infection that is gentler with respect to the oral microflora and which is more effective, due to the extended dwelling time of the product.

The biguanides constitute highly effective antimicrobial substances with a broadband effect vis-à-vis a great number of microorganisms. They also have a good effect vis-à-vis multiresistant agents. Biguanides offer not only good antibacterial action, but also very low cytotoxicity. In this context, polyhexanite is expediently used as a concomitant substance in the amount of 0.5 to 1%, relative to the dry content of the collagen matrix.

Chlorohexidine is expediently used in the amount of 0.5 to 1.5%, specifically approx. 1%, relative to the dry content of the collagen matrix. The same applies to chlorohexidine. Octenidine, taurolidine, and cetylpyridinium hydrochloride are also expediently used in the amount of 0.5 to 1.5%, specifically in the amount of approx. 1%, relative to the dry content of the collagen matrix.

According to the invention, the concentration of the antimicrobial compounds used as concomitant substance is expediently a total of 0.5 to 2%, relative to the dry weight of the carrier material, for purposes of antimicrobial effectiveness.

The antimicrobial compounds can also include compounds containing silver. They may be a soluble silver salt, a silver salt of low solubility, silver complexes, organic silver compounds and/or elementary silver, preferably silver chloride. These compounds containing silver may be present as silver nitrate, silver acetate, silver carbonate, silver halogenide, [Silbersulfatiacin], silver diamine complexes, silver dithionate complexes, silver thiocyanate, silver powder and/or mixtures thereof.

Silver ions show good antimicrobial action in the treatment of infected wounds. Even in low concentrations (1×10-6 to 1×10-9 mol/l), silver ions are highly effective in combating bacteria, viruses and parasites. In this context, silver products with low solubility are frequently used, resulting in small amounts of silver ions being released. These substances include silver chloride, silver bromide, silver iodide, silver oxide, silver carbonate, silver thiocyanate and also silver powder. Following their application, these substances continuously release small amounts of silver from the pad. The silver ions are reduced to silver as a result of their antimicrobial effect. This causes a rapid reduction of the concentration of the silver ions in the wound fluid. Since only silver ions have an antimicrobial effect, it is essential that silver ions be available for an extended period of time in sufficient concentration. Thanks to the use of silver salts of low solubility, silver ions are continuously redelivered to preserve the chemical balance, for as long as silver salts are still available in the wound pad.

In the beginning of the treatment, an infected wound requires a larger amount of silver, and in the subsequent course, after the microbial count has been reduced, it requires a continuous release of silver ions. In accordance with the invention, this can be achieved using a mixture of silver complexes and silver compounds of low solubility in the polymer matrix.

Here, the silver complexes are first dissolved out of the collagen matrix. This results in a burst release of silver into the wound fluid. The addition of silver salts of low solubility makes sure that a continuous release of silver ions occurs over a long period of time. In one embodiment, 5 ml of a silver acetate solution is added to 1000 ml of a 1% collagen matrix solution (collagen and other excipients or additives). The solution contains 15.46 mg silver acetate. Following homogenization of the solution, 5 ml of a sodium chloride solution was added. The solution contained 5.4 mg sodium chloride. In the alternative, it is also possible to first mix the silver acetate solution with the sodium chloride solution; then this mixture is added to the collagen dispersion. The subsequent addition of 29.85 mg silver dithionate (Ag[S2O3]2) is possible. Silver concentration: 0.1% AgCl and 0.1% silver complex.

The concentration of the compounds containing silver is expediently in the range of 0.1 to 2%, relative to the dry weight of the carrier material, to achieve the antimicrobial effectiveness.

As explained previously, the bioresorbable material expediently includes a finely pored sponge, which makes it possible to obtain an especially favorable cushioning effect. Due to its pore structure, the collagen matrix, preferably used as bioresorbable material, absorbs a lot of fluid. Furthermore, the foam-like structure results in good compressibility. The foam is therefore especially suited to fill out uneven or ragged sites. A dry collagen sponge spontaneously adheres to the gums or the teeth. It has been shown empirically that a film-like material includes markedly reduced adhesion in applications in the oral region.

In order to achieve a satisfactory cushioning effect with simultaneous prevention of an unpleasant filling effect, it has been shown that it is expedient for the bioresorbable material to include a foam or a sponge which includes a thickness of 0.5 to 4 mm in the Z direction (cf. FIG. 11).

Expediently, the foam is formed in special shapes for dental applications.

It has been shown to be especially advantageous when the product in accordance with the invention is capable of being sterilized by radiation (25 kGy) or by means of exposure to ethylene oxide.

With respect to the desired cushioning effect of the product in accordance with the invention, it has proven to be advantageous when the bioresorbable material is compressible, using brief exertion of a pressure of 1 bar in the Z direction (cf. FIG. 11), to less than 50%, preferably less than 40%, specifically 25% or less, of the original thickness.

In addition, or in the alternative, the bioresorbable material in accordance with the invention may also display good plasticity in the X and Y direction (cf. FIG. 11), preferably due to a lyophilization process.

Within the context of compatibility of products in accordance with the invention, it is intended for the bioresorbable material to be degradable in dental applications and to be completely resorbed in the event that it is accidentally swallowed. To increase its durability, the biodegradability of the bioresorbable material may be reduced by at least partial crosslinking. It has been found to be expedient when the bioresorbable material is capable of being crosslinked with bifunctional or multifunctional aldehydes, carboxylic acids, carboxylic acid chlorides, carboxylic acid aldehydes and/or epoxides. In addition, or in the alternative, the biodegradability of the bioresorbable material may be expediently modified by means of cleavable bridges as well as γ radiation or β radiation.

As can be gathered from the above explanation of products in accordance with the invention, the bioresorbable material is used for wound healing and/or disinfection (e. g. treatment of oral sores, pressure sores, mycoses, canker sores and other infections) within the framework of the invention. For this purpose, the bioresorbable material may be used as a pad between dental prostheses or braces and the oral mucosa, a wound or the gums. In addition, or in the alternative, the bioresorbable material can also be used as adhesive medium for complete or partial prostheses, where it can possibly be used, if applicable, with a chitosan additive for the hemostasis of bleedings in dental applications (even with individuals under the influence of anticoagulants, such as e. g. Marcumar).

### BRIEF DESCRIPTION OF THE DRAWINGS

In the following, the invention will be explained in more detail with reference to the drawing, to which explicit reference is made with respect to all details that are essential to the invention and that are not elaborated on in the description.

FIG. 1A shows a product in accordance with the invention for application in conjunction with complete prostheses in the lower jaw;

FIG. 1B shows a product in accordance with the invention for application in conjunction with complete prostheses in the upper jaw;

FIG. 2 shows a product in accordance with the invention for the treatment of gums;

FIGS. 3A and 3B show a collagen sponge with a string, to be attached e. g. to a tooth;

FIG. 4 shows a cut-in collagen sponge, which can be torn off;

FIG. 5 shows a collagen strip which can be used to produce products in accordance with the invention;

FIG. 6 shows a product in accordance with the invention, in the form of a U-shaped collagen sponge for the treatment of a tooth;

FIG. 7 shows a dispenser for the provision of products in accordance with the invention;

FIG. 8 shows a product in accordance with the invention for the management of the gums on top of several teeth;

FIG. 9 shows a product in accordance with the invention for application in conjunction with complete prostheses in accordance with another embodiment of the invention;

FIG. 10 shows a product in accordance with the invention in an oval shape for variable uses; and

FIG. 11 shows a 3D view of the biodegradable material that can be used in accordance with the invention.

### DETAILED DESCRIPTION OF THE EMBODIMENTS OF THE INVENTION

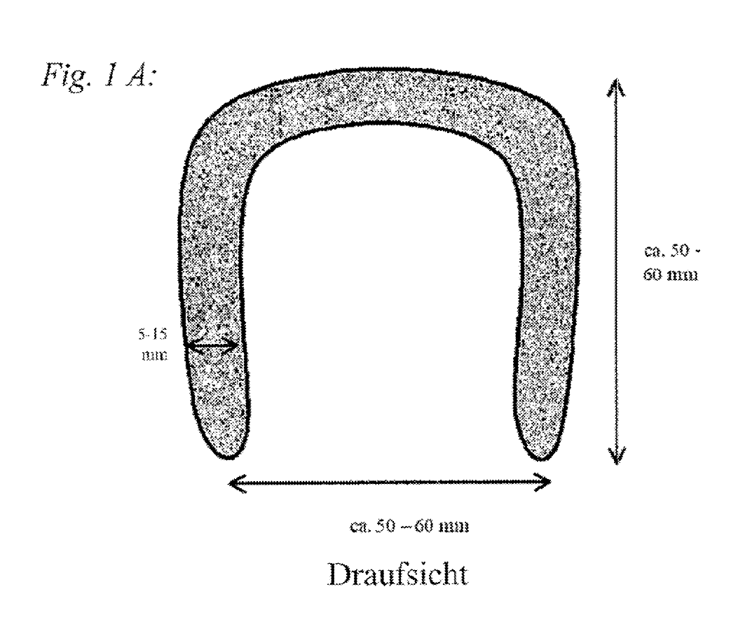

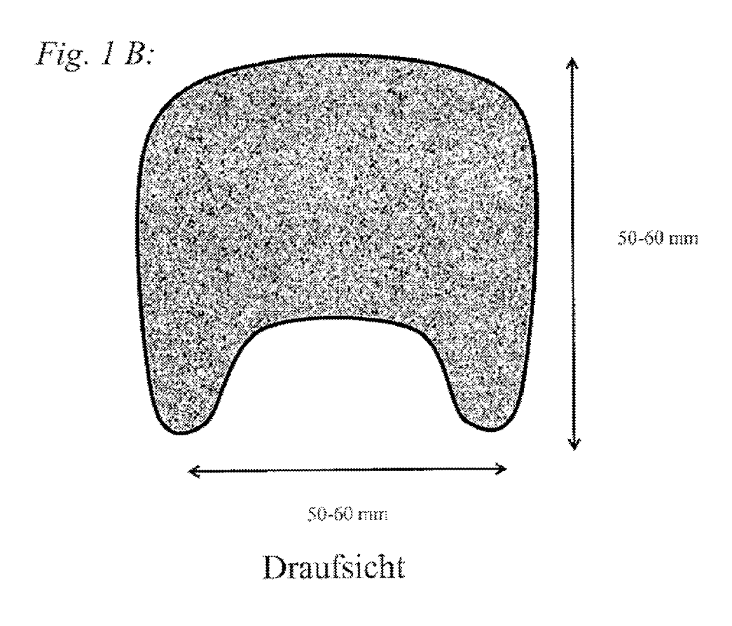

The product shown in FIG. 1A for use in conjunction with a complete prosthesis in the lower jaw essentially has a U-shaped design. It has a width between the legs of approximately 50 to 60 mm and a height of approx. 50 to 60 mm as well. The material strength of the legs is approx. 5 to 15 mm.

With respect to the outer shape, the product shown in FIG. 1B corresponds essentially to the product shown in FIG. 1A. But it is filled in between the legs so that it is capable of virtually completely filling in the space between a complete prosthesis in the upper jaw and the palate.

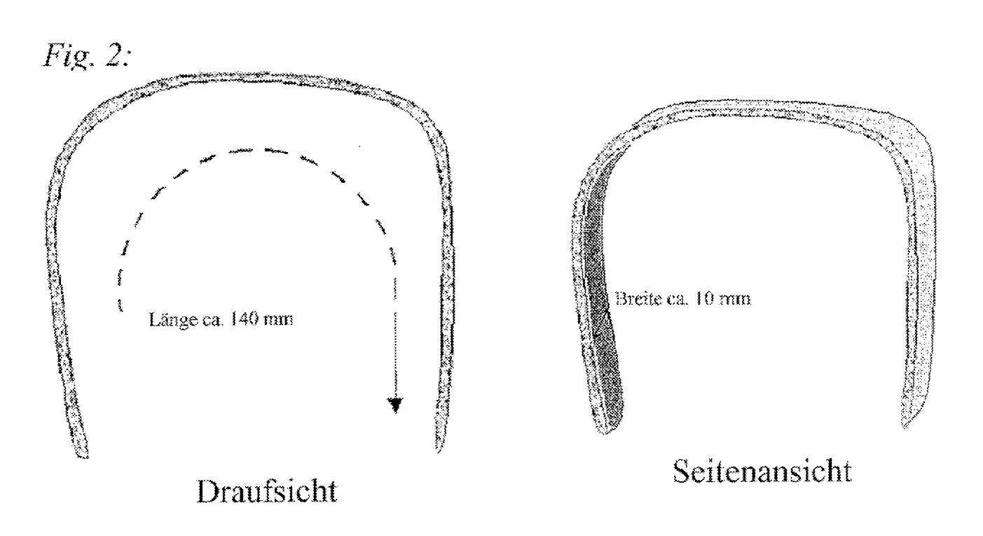

The product in accordance with the invention shown in FIG. 2 for the management of the gums also essentially has a U-shaped design. It has a length of approx. 140 mm, with the material strength being approx. 10 mm in accordance with the side view.

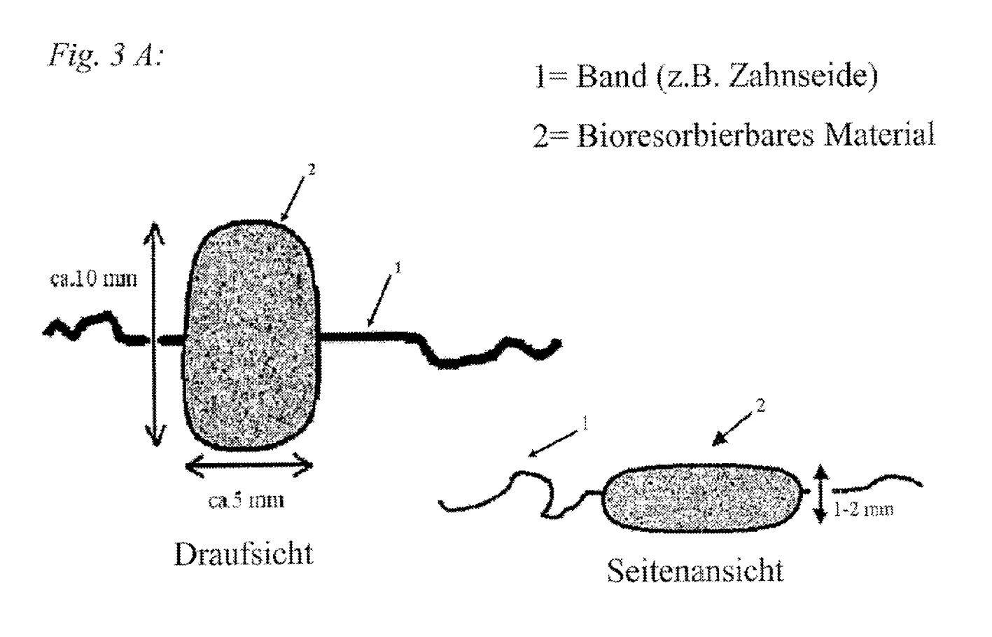

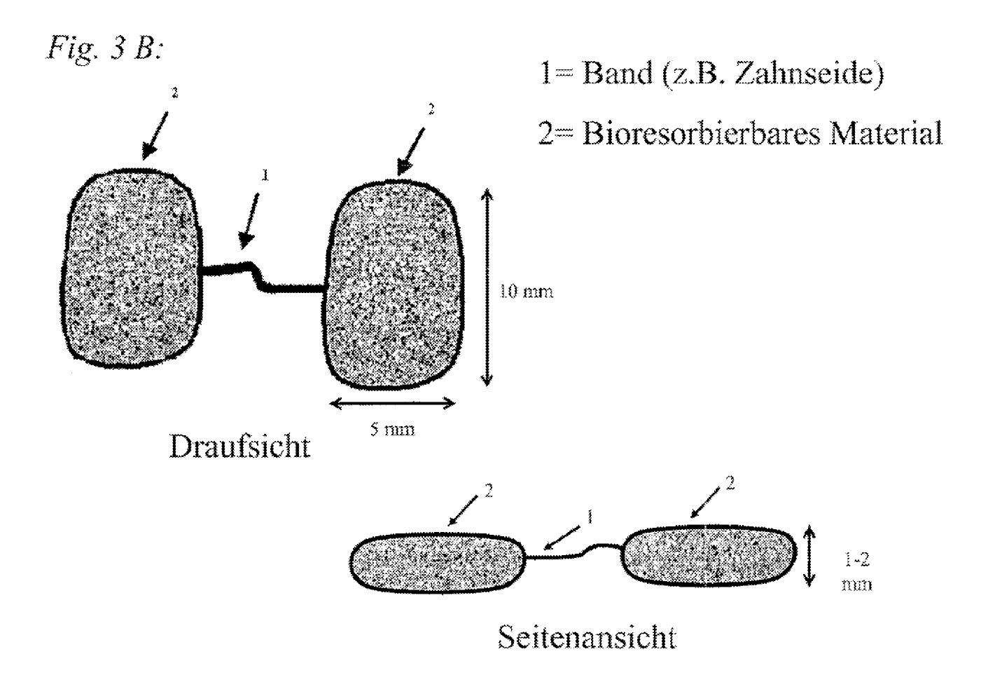

The product shown in FIG. 3 for the management of several teeth includes a collagen sponge with a height of approx. 10 mm and a width of approx. 5 mm, with the thickness of the collagen sponge being approx. 1 to 2 mm. As can be seen especially clearly in FIG. 3A, attachment strings are attached to the collagen sponge. These strings may be made of dental floss, for example. In accordance with FIG. 3B, it is also possible for two or more collagen sponges of the type shown in FIG. 3A for the management of various areas of a tooth and/or for the management of adjacent teeth to be interconnected by means of attachment strings made, for example, of dental floss.

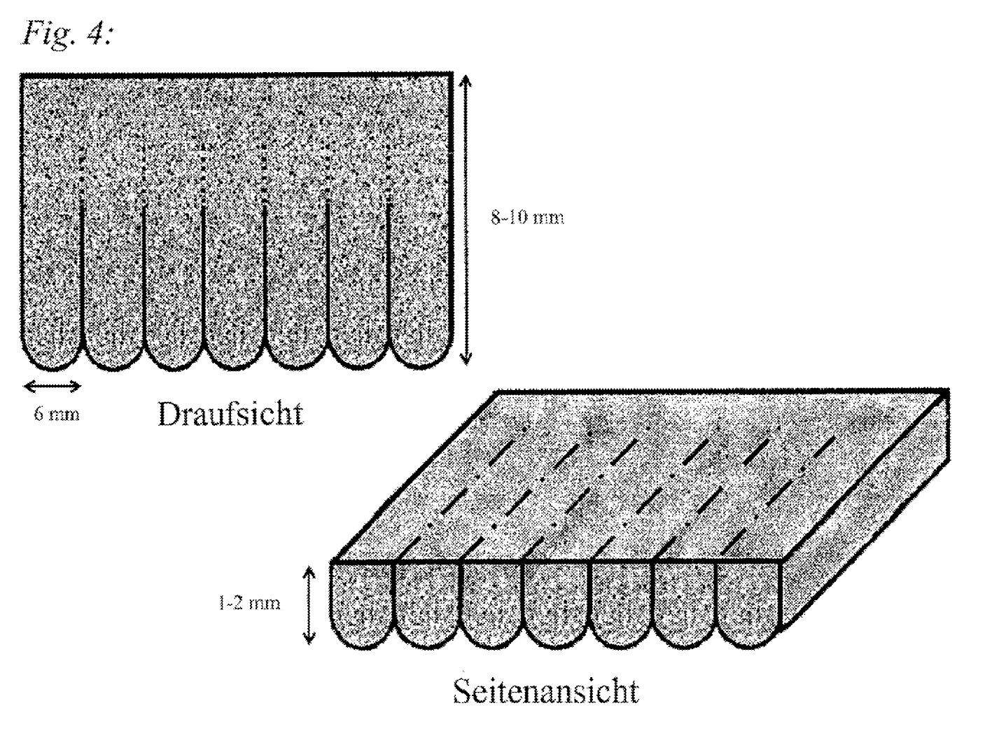

The cut-in collagen sponge for the production of products in accordance with the invention shown in FIG. 4 is a collagen strip of any length, and a width of approx. 8 to 10 mm and a thickness of approx. 1 to 2 mm. As can be seen especially clearly in the side view, this collagen strip displays cuts extending vertically to the longitudinal direction and having a distance of approx. 6 mm from each other.

The strip thus prepared may be used, for example, to produce the products shown in FIG. 3.

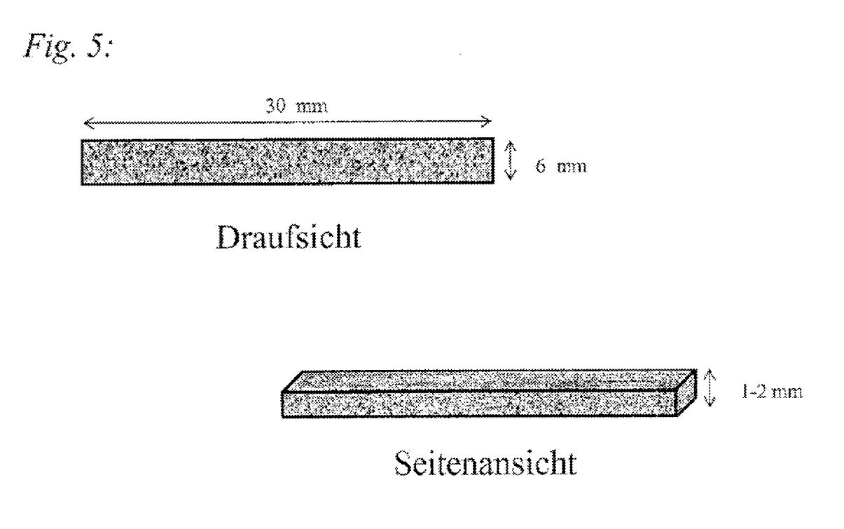

The collagen strip shown in FIG. 5 may be used, for example, to produce the products in accordance with the invention shown in FIG. 2.

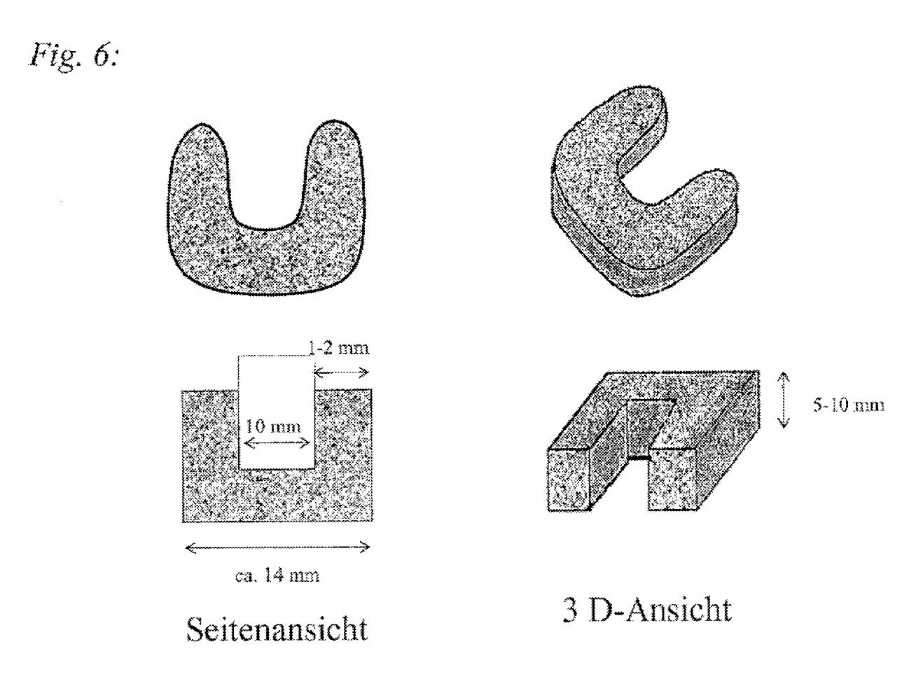

In addition, or in the alternative, the collagen strip shown in FIG. 5 can also be used to produce the products shown in FIG. 6.

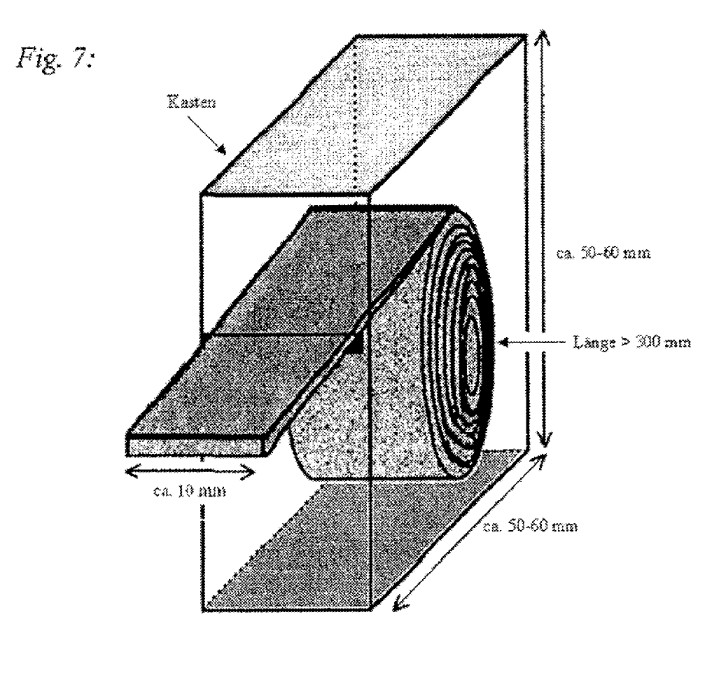

The collagen strip shown in FIG. 5 can, for example, be spirally wound up and housed in a dispenser in accordance with FIG. 7.

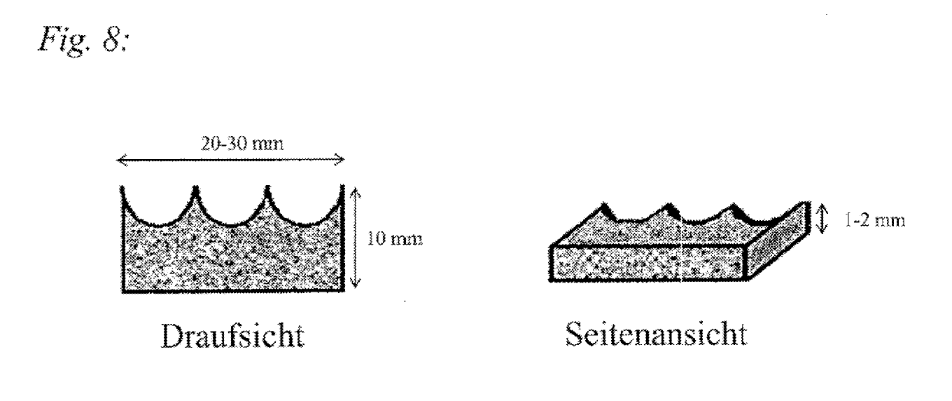

The product shown in FIG. 8 for the management of the gums on top of several teeth has wave-like shape at the longitudinal edge of the strip, so as to enable its adjustment to the tooth indents in the gums.

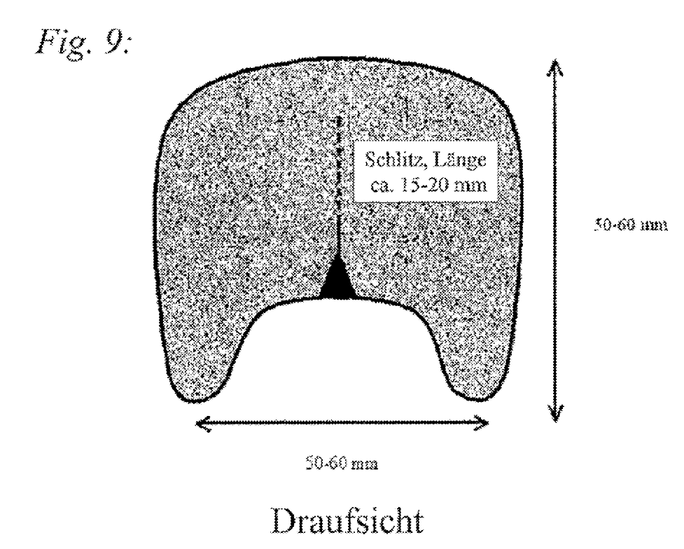

The product shown in FIG. 9 is a special form of the product whose general form is shown in FIG. 1B, with said special form allowing, thanks to a cut, an improved adjustment to the prosthesis on the upper jaw.

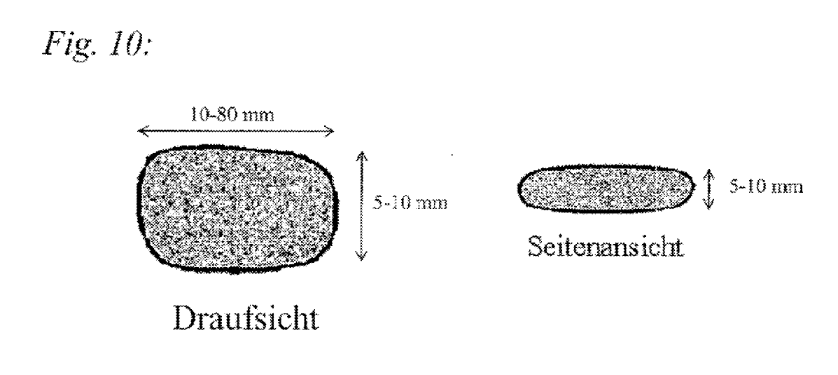

The product shown in FIG. 10 can be universally used in the oral cavity.

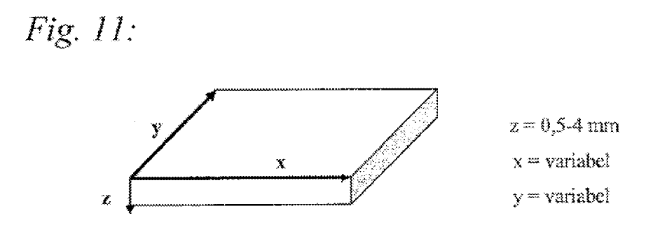

The product shown in FIG. 11 serves to explain the above-described compressibility of the products in accordance with the invention.

The dimensional data pointed out above and specified in the drawing are preferred ranges within the framework of the invention. It is, however, possible to deviate from these ranges. This specifically applies to the application of the products in accordance with the invention in children. In this application, the shapes shown in the drawing may be preserved but the dimensions are respectively reduced.

It was already pointed out that the bioresorbable material used in accordance with the invention shows good adhesion to the oral mucosa. This particularly applies to bioresorbable material in the form of collagen. When a pad containing collagen is placed in the oral cavity, it adheres very well to the teeth and gums. The following tests were performed for the quantitative determination of its adhesion:

A slide was coated with a 2.5% warm agar solution. The agar solution was completely dried. The samples to be analyzed were moistened with saliva and added to a coated slide. A second coated slide was moved close to the sample after the sample was treated with saliva (distance: 9.6 mm). A contact of at least one minute was maintained before a tension measurement was performed. The sample includes an area of 15 mm×20 mm. The tractive force required to detach the sample from the slide (adhesion) is presented in the following table:

| Name | F max [N] |
|---|---|
| Subrasorb C (reference) | 4.3 |
| Collagen + 50% glycerin | 2.3 |
| Collagen + 20% PEG | 1.78; 1.54; 1.65 |
| Collagen + 10% Na alginate | 5.2 |
| Collagen + 10% gellan | 5.0 |
| Collagen + 10% [polyacrylic] acid | 2.3 |
| Collagen + 60% honey | 7.2 |
| Collagen + 10% Na− + 10% Ca | 4.5 |
| alginate | |
| Collagen + 10% CMC | 1.8 |
| Collagen + 60% honey + 5% propolis + | 3.7 |
| 10% Na alginate | |

Accordingly, the product according to the invention or the bioresorbable material of the product according to the invention expediently has an adhesive capacity of 1 Newton or more, specifically 2 Newton or more, and especially preferably 4 Newton or more.

Furthermore, tests to determine the compressibility of various collagen pads were performed. To this end, the collagen pads (bioresorbable material) were loaded for 10 seconds with a pressure of 1 bar. The thickness before and after the loading was determined.

In addition, the sample was firmly compressed between the index finger and thumb for several seconds, in order to determine the maximum compressibility of a dry pad. The results are presented in the following table:

| | | Thickness, | | Thickness | |
|---|---|---|---|---|---|
| | Thickness, | 10 sec. | | 10 sec. | |
| Name | no load | 1 bar | | >5 bar | Comments: |
| Subrasorb C | 7.1 mm | 1.7 | mm | 0.35 mm | reversible |
| (reference) | | | | | |
| Collagen + 30% | 6.9 | 0.6 | | 0.24 | irreversible |
| glycerin | | | | | |
| Collagen + 50% | 5.7 | 0.38 | | 0.26 | irreversible |
| glycerin | | | | | |
| Collagen + 10% | 6.9 | 1.1 | | 0.48 | irreversible |
| propolis | | | | | |
| Collagen + 10% | 6.0 | 1.1 | | 0.31 | irreversible |
| CMC | | | | | |
| Collagen + 60% | 6.0 | 0.85 | | 0.29 | irreversible |
| honey | | | | | |
| Collagen + 10% | 5.0 | 0.9 | | 0.2 | irreversible |
| polyacrylic | | | | | |
| acid | | | | | |
| Collagen + 10% | 7.0 | 0.9 | | 0.34 | irreversible |
| gellan | | | | | |
| Collagen + 10% | 6.0 | 0.8 | | 0.27 | irreversible |
| Na alginate | | | | | |
| Collagen + 10% | 6.1 | 0.33 | | 0.20 | irreversible |
| Na alginate + | | | | | |
| 60% honey + | | | | | |
| 5% propolis | | | | | |
| Collagen + 20% | 6.0 | 1.5 | | 0.31 | reversible |
| PEG | | | | | |
| Collagen + 10% | 7.0 | 1.2 | | 0.45 | reversible |
| Na− + 10% | | | | | |
| Ca alginate | | | | | |

The reference product specified in the table, “Suprasorb C,” is a product that was made in accordance with DE 38 32 162 C2.

1-76. (canceled)

77. A product for the management of inflammations, pressure sores and/or oral sores (aphthas), the product comprising a bioresorbable material, wherein the bioresorbable material includes a flexible and/or compressible pad element that adheres to an oral mucosa and includes a collagen and an alginate, said product having a U-shaped design for use in conjunction with a lower jaw and/or upper jaw, wherein the bioresorbable material is mixed with a polycation agent as a concomitant substance.

78. The product in accordance with claim 77, wherein at least a portion of a space between legs of the U-shaped design is filled, so that the product is capable of filling in a space between a complete prosthesis in the upper jaw and a palate.

79. The product in accordance with claim 77, wherein a distance between the legs is 50 millimeters (mm) to 60 mm.

80. The product in accordance with claim 77, wherein a height of the U-shaped design is 50 millimeters (mm) to 60 mm.

81. The product in accordance with claim 77, wherein a thickness of the legs is 5 millimeters (mm) to 15 mm.

82. The product in accordance with claim 80, wherein the height is a dimension that extends in a direction that is parallel to an extended direction of the legs.

83. The product in accordance with claim 81, wherein the thickness is a dimension that extends in a direction that is perpendicular to an extended direction of the legs.

84. The product in accordance with claim 77, wherein the bioresorbable material further includes at least one bioresorbable polymer from a group consisting of gellan, chitosan, chitin, k-carrageenan, φ-carrageenan, xanthan, carboxymethyl cellulose, hydroxyethyl cellulose and hydroxy ethyl propyl cellulose.

85. The product in accordance with claim 77, wherein the bioresorbable material is capable of being compressed to less than 50% of its original thickness.
